(19) United States
(12) Patent Application Publication
(10) Pub. No.: US 2023/0392176 A1
(43) Pub. Date: Dec. 7, 2023

(54) Method of purifying mRNA using substrate dependent ribozyme and solid-support attachment tag

(71) Applicant: Hari Bhaskaran, oceanside, CA (US)

(72) Inventor: Hari Bhaskaran, oceanside, CA (US)

(21) Appl. No.: 18/072,674

(22) Filed: Nov. 30, 2022

Related U.S. Application Data
(60) Provisional application No. 63/349,190, filed on Jun. 6, 2022.

(51) Int. Cl. C12P 19/34

(52) U.S. Cl. CPC C12P 19/34

(57) ABSTRACT

The current invention is a method for generating mRNA and purifying it from impure preparations that result from processes used to generate the mRNA. The processes may include the commonly used cell-free, enzymatic synthesis (in-vitro transcription, IVT) or may include preparation of crude cell extracts that contain mRNAs. The key steps in purification involves capture of the mRNA via base-pairing of a covalently attached adapter RNA to the chromatography column, and subsequent on-column cleavage of a substrate-dependent ribozyme, also covalently attached, which cuts and releases the mRNA upon addition of a specific substrate.

### RELATED APPLICATIONS

This application claims the benefit of provisional patent application U.S. 63/349,190, filed Jun. 6, 2022.

### FIELD OF THE INVENTION

The present invention applies to the field of RNA production technologies in general, and relates to mRNA production in particular. The present invention also relates to mRNA purification strategies that follow production which in turn may encompass any methodologies used to generate RNA, either cell-free systems or cell-based systems. Unlike a traditional RNA synthesis system such as in vitro transcription (IVT), wherein a DNA template is designed to encode only the RNA of interest, the present invention encodes a substrate-dependent catalytic RNA (ribozyme) and an adapter sequence in addition to the RNA of interest. While the adapter helps the RNA of interest bind to the chromatography column matrix via a short complementary DNA oligo, the ribozyme cleaves (cuts) the mRNA at the desired location upon addition of specific substrate at the desired time. The RNA of interest is thus liberated from the ribozyme and adapter upon cleavage. The present invention offers both spatial and temporal control over RNA purification. The purification strategy is aimed at significantly reducing both product related and process related contaminants in a single step without the need for multiple chromatography steps.

Safety, efficacy and stability of mRNA depend highly on the purity of mRNAs. IVT process for mRNA synthesis requires input of several raw materials such as ribonucleotides, enzymes and plasmid DNA. Following IVT synthesis of mRNA, unreacted raw materials are process related impurities. For cellular transcription, host cell DNA, RNA and proteins are source impurities that must be removed. Finally, double stranded RNA (dsRNA), short truncated mRNAs and RNA aggregates are product related impurities which may arise following IVT or cellular transcription and must be eliminated during a successful purification.

In particular, the present invention offers the possibility of completely eliminating a specific contaminant known as dsRNA that is known to trigger undesirable immune responses in patients[1].

### BACKGROUND OF THE INVENTION

mRNAs have gained considerable attention in recent years, particularly because of the success of mRNA-based Covid-19 vaccines. By now (November 2022), mRNA-based Covid vaccines have been successfully administered to hundreds of millions of people in a safe, efficacious manner[2, 3]. The speed of development of these vaccines are unparalleled in the history of vaccines, thanks to the facile methods used in the production of mRNAs. Enzymatic synthesis of mRNAs by in vitro transcription (IVT), using a specific DNA template that encodes the desired protein, followed by purification using affinity chromatography has been the gold-standard for the synthesis and purification of mRNAs[4].

The recent success with Covid-19 vaccine is a testament to the simplicity and ease of manufacturing RNAs using the IVT process. While IVT can generate sufficient amounts of good quality RNA for vaccine production (10 to 100 ug dose per patient—Pfizer/Biontech, Moderna), it becomes expensive to generate vast quantities of high-quality mRNAs that are needed for other applications such as bio-therapeutics (example: protein replacement, monoclonal antibody and cytokine therapies), where the requirements are high (milligram amounts) per patient and also necessitates repeat dosing[5]. In addition, the quality of the mRNA becomes very important at high and repeated dosage[6]. IVT generates product related and process related impurities and these have to be efficiently removed prior to formulation as a drug product[7-11].

Currently, multiple chromatographic methodologies are employed to remove these contaminants leading to increased production costs[7, 11]. An efficient, single process of purification that removes all of these contaminants is highly sought because this would not only reduce the production costs but also tremendously improve the purity of the resulting mRNAs. This is because frequent handling and processing of mRNAs lead to partial degradation and result in shorter fragments of mRNAs. These fragments are contaminants that chemically resemble the full-length mRNAs and therefore difficult to eliminate, once generated.

mRNAs are generally obtained through in vitro transcription (IVT) wherein the RNA of interest is synthesized using a virus-derived polymerase such as T7 RNA polymerase using raw materials such as DNA template, nucleotide triphosphates (NTPs) and magnesium. After synthesis, the raw materials used in IVT must be removed using chromatography purification methods.

### BACKGROUND OF THE INVENTION

Currently, mRNA is generated in large quantities by IVT of DNA template using T7 RNA polymerase, where the mRNA simply falls off the template strand once it reaches the end of the template. This process results in an identical copy of the non-template strand but in the form of poly ribonucleotides instead of poly deoxy-ribonucleotides of DNA. The poly-A tail at the 3′ end is either encoded or less frequently, added post-IVT using a polyA polymerase.

Capping can be co-transcriptional using a capping analog such as anti-reverse capping analog (ARCA) (or) using Cleancap® (Trilink® biotechnologies). Capping can also be performed post-IVT using a vaccinia capping system[4]. Following IVT, capping and poly-A tail addition, the mRNA is purified by affinity chromatography such as oligo(dT) or anion exchange. Among affinity methods, oligo(dT) method has shown to be superior over anion exchange because of the ability to capture only poly-adenylated mRNAs; short truncated mRNAs are mostly excluded. mRNAs generated by IVT contain a significant amount of product-related and process-related contaminants, some of which require one or more chromatography steps in addition to affinity purification methods. These may include cellulose column chromatography and SDVB column chromatography for removing dsRNA[12]. Hydrophobic interaction chromatography (HIC) for removal of endotoxin[13]. These additional purification steps can be expensive, lead to loss of mRNA yields, poor recovery and mRNA degradation. There needs to be a strategy for production and purification that offers much better purity in a single step compared to existing methods, particularly with respect to removal of dsRNA contaminant. Additionally, there is currently no way to generate homogenous mRNA of precise length because the run-off transcription reactions are prone to produce a varying amounts of short-length mRNA molecules in addition to the predominant full-length mRNA molecule. This is particularly problematic for mRNAs that are 5000 nucleotides and longer, as longer mRNA syntheses produce greater amounts of truncated mRNAs. While the problem is a result of an inherent consequence of run-off transcription (IVT), a system of synthesis that produces homogenous mRNAs with uniform sizes is imperative for successful transition of long-length mRNAs into the clinic.

### SUMMARY OF THE INVENTION

We have designed an mRNA purification process and devised a purification strategy that would substantially remove process and product-related impurities in a single step. In particular, the current invention not only decreases a key contaminant—dsRNA impurity—substantially but also produces homogenous mRNA with uniform mRNA molecules. This process will also form the basis of a general production strategy for overexpression and purification of mRNAs directly from eukaryotic cells such as yeast, making mRNA production even cheaper and thereby substantially reducing the cost of mRNA therapy for patients worldwide.

The process involves addition of two sequence elements into the DNA construct encoding the RNA, immediately after the 3′ end of the desired mRNA (mRNA of interest) to be purified—a ribozyme that cleaves at its 5′ end and an adapter sequence for the capture of the mRNA-ribozyme-adapter RNA to the column chromatography matrix (FIG. 1). The RNA that this whole construct encodes is written as mRNA-ribozyme-adapter and given the name ‘cistRNA’ (FIG. 2a). The whole RNA is then captured by base pairing of adapter sequence to the DNA (single-stranded) that is in-turn immobilized on the chromatography column (FIG. 2b). After capture of cistRNA, the ribozyme part of the mRNA is allowed to fold in the presence of Mg²⁺ ions (FIG. 2c). Addition of specific substrate for the ribozyme then cleaves (cuts) just after the mRNA of interest (FIG. 2d). Further elution with buffer releases the mRNA of interest from the column matrix (FIG. 2e). This method will be hereby known as SPecific Capture and Cleavage technology or simply abbreviated as ‘SPECC’. This results in purified mRNA (FIG. 3a) with reduced dsRNA (FIG. 3b).

### BRIEF DESCRIPTION OF THE DRAWINGS

FIG. 1: Schematic of the DNA encoding RNA elements (cistRNA) for synthesis and purification

FIG. 2: Schematic of RNA purification following synthesis of full-length ‘cistRNA’
(a) Binding of cistRNA is facilitated by addition of monovalent (Nat) ions
(b) Addition of Mg²⁺ ions cause ribozyme to fold only without cleavage of the cistRNA
(c) whereas addition of Mg²⁺ ions along with substrate, glucosamine-6-phosphate or imidazole (indicated by the circle), causes folding and cleavage of the RNA (indicated by ‘x’)
(d) Cleavage of full-length ‘cistRNA’ results in liberation of mRNA of interest (e).

FIG. 3: Cisterna mRNA (post-cleavage) shows less dsRNA compared to uncleaved mRNA (cistRNA). ‘L’ represents DNA ladder and its used here for imparting general appreciation for differential sizes and not for sizing application of RNA.

### DETAILED DESCRIPTION

### Definitions

1) cistRNA: mRNA construct described in the present art that includes a ribozyme sequence and an adapter sequence at its 3′ end
2) Chromatography resin: may denote any matrix that bind directly to mRNA or may bind to a complementary DNA oligo. The resin may be made of cellulose or propriety magnetic particle covalently attached to a ligand such as streptavidin or an antibody.
3) Chromatography monolith column: may denote any matrix that is not a resin but based on a composite or matrix that is immobile and may allow large particles to flow through
4) Cleavage: refers to breaking of the covalent bond of the RNA molecule upon activation of the ribozyme by the substrate Elution: refers to collection of purified mRNA off the chromatography column
6) ORF: Open reading frame—encodes protein

Described herein are strategies, that include sequence additions, methods and chromatography processes for synthesizing and purifying mRNAs from contaminating nucleotides, host genomic DNA, RNA and proteins. The sequence additions, methods and purification processes described herein, are particularly suited to efficiently remove one product related contaminant known as double stranded RNA or dsRNA. The sequence additions, methods, and processes described herein are also suited to specifically capture only mRNAs expressed from transfected plasmids and not the endogenous mRNAs from cells. Thus, the sequence additions, methods, and processes pave the way for a new type of producing mRNAs compared to the older IVT method. mRNAs can be synthesized directly in cells from plasmids using specific templates, captured using a complementary DNA sequence and cleaved by the ribozyme after capture to release the desired mRNA.

The advantages of producing mRNAs in Eukaryotic cells such as Yeast is based on the findings that mRNAs synthesized in vivo are different albeit superior from IVT mRNAs[14-16]. Briefly, these mRNAs are likely to have structures and modifications similar to the ones that occur naturally in higher eukaryotes such as humans. The ability to produce mRNAs in yeast also implies that high costs associated with raw materials such as capping reagents and modified nucleotides are also negated.

The sequence additions and method disclosures are not limited to the particular embodiment of the disclosure described herein, but may encompass several variations of the particular embodiment. Thus, variation in sequence of the ribozyme, capture sequence and steps leading up to and including substrate-dependent ribozyme cleavage still fall within the scope of the approved claims.

Described herein are the sequence designs of the template, be in linearized plasmid (or) a PCR amplified template for the in vitro synthesis of mRNA. The sequence design could also be incorporated into an intact plasmid for expression of mRNA in cells.

The template strand, as in either linearized plasmid or PCR amplified template, is transcribed into RNA using an enzyme such as T7 RNA polymerase[17]. The resulting sequence of the RNA is similar to the non-template strand except it's made of ribonucleotides instead of deoxyribonucleotides of DNA.

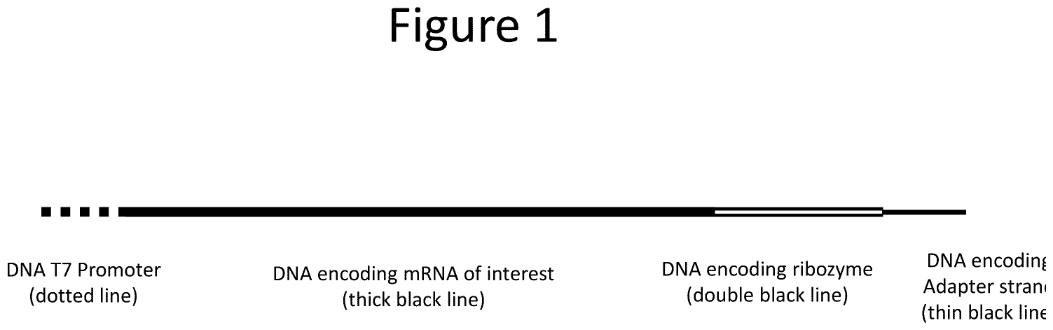

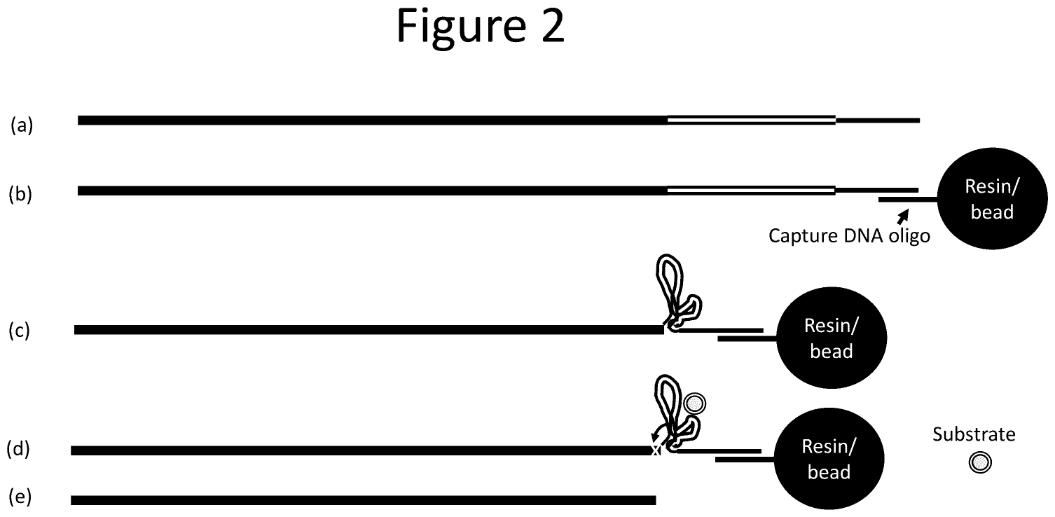

Referring to FIG. 1, the order of DNA sequence encoding the cistRNA from 5′ end to 3′ end (from left to right) is mRNA-ribozyme-adapter where mRNA is further composed of cap-5′ untranslated region (5′ UTR)-ORF-3′ untranslated region (3′ UTR)-polyA from the 5′ end to the 3′ end. The 5′ UTR may or may not be followed by a Kozak sequence that is known to enhance translation but not necessary. ORF is open reading frame that could be any protein coding RNA. Ribozyme comprises any substrate-dependent catalytic RNA that can precisely cleave or in other words, break the covalent bond between two nucleotides. This results in two RNAs with 5′ hydroxyl group at the ribozyme end and a 2′-3′ cyclic phosphate at the 3′ end of the RNA of interest.

The adapter RNA comprises any sequence of RNA and not a poly(A) as described in previous patents for capture of mRNA with polyA tails[18, 19]. In a preferred embodiment, the sequence is a poly (CA)x and in another embodiment the sequence is a poly (UA)x, where x is a whole number between 1 and 50. However, any sequence other than a polyA and attached covalently to the ribozyme and used to specifically bind a capture DNA oligo on the chromatography column matrix fall within the scope of the appended claims.

The capture oligo is a DNA whose sequence is complementary to the adapter sequence and used to capture the whole RNA construct as described below. The capture oligo contains a linker and a complementary capture sequence. The capture oligo is either covalently attached to a column matrix or biotinylated at its 5′ end via the linker. The biotinylated oligo interacts strongly with streptavidin coated resin beads of a chromatography matrix.

The capture oligo may also be attached to a monolith matrix, a cellulose resin, magnetic beads or any other chromatography matrix by either covalent bonds, strong electrostatic or electromagnetic interaction, hydrophobic or hydrogen bonds. In one embodiment, biotinylated DNA oligo is pre-bound to the streptavidin chromatography matrix. In another embodiment, it is covalently attached by activation of a carbonyl group to a primary amine as in the previously described art[19].

The capture sequence of the capture oligo may not be an oligo dT as in the previous art[19], but any other sequence variation complementary to the adapter sequence described. In one preferred embodiment, it is poly (GT)x where x denotes a whole number between 1 and 50. The capture oligo may also contain a linker as described in previous art, however it may not be a poly cytidine[19].

In a preferred embodiment, the linker part of the capture oligo may be a poly (dA)x or a poly (dG)x but may not be a poly dC or poly dT where x denotes a whole number between 0 and 50. Selection of the length or sequence of either the capture sequence or linker is entirely within the scope of ability of a skilled person.

Described herein is the method of preparing one embodiment of the chromatography resin that is comprised of streptavidin magnetic beads bound by biotinylated capture oligo. Sufficient amounts of streptavidin beads to bind the required amount of capture oligo were added to a microfuge tube. In one embodiment, 125 microliters containing 500 micrograms of beads were added to the tube and diluted two-fold with 125 microliters of wash/bind buffer containing 20 mM Tris pH 7.0, 500 mM NaCl and 1 mM EDTA, vortex mixed to resuspend. The tube was inserted into a magnetic stand and the beads were allowed to collect to the side of the tube for 30 seconds before the supernatant was removed and discarded. In a preferred embodiment, one nanomole of biotinylated capture oligo containing either a (A)₁₄ linker or (G)₁₄ linker but not a (C)ₓ linker and a poly (GT)10 capture sequence was mixed with the beads in a buffer containing 20 mM Tris pH 7.0, 500 mM NaCl and 1 mM EDTA. The beads were then incubated at room temperature for 5 minutes with occasional vortex mixing. The beads were then washed with 125 uL of the wash/bind buffer by adding the buffer, mixing well, applying to magnetic stand and removal/disposal of the supernatant. The wash was performed twice before the chromatography resin is ready for purification.

In one embodiment, 500 nanograms of mRNA-ribozyme-adapter is mixed with the prepared beads in the presence of 25 to 250 millimolar Tris pH 7.0 and 100 to 500 millimolar monovalent ions such as sodium, potassium, lithium, ammonium and any other ions holding a net positive charge of one. The counter ions may be chloride, acetate or any other ions holding a net negative charge of one. In one preferred embodiment, Tris concentration was 25 millimolar and monovalent ions was sodium chloride at a concentration of 250 millimolar final concentration. In one embodiment, binding was performed in the presence of monovalent ions although divalent ions may substitute for the monovalents. The RNA is mixed well with the beads and incubated for x minutes, where x may denote any time between 5 and 30 minutes to allow binding of all of the mRNA-ribozyme-adapter RNA by base-pairing of the adapter part of RNA to the capture oligo on the beads. In a preferred embodiment, binding was performed for 10 minutes.

Also described herein, and shown in FIG. 2, is the method for capture of mRNA-ribozyme-adapter (cisterna) to chromatography resin followed by folding of the ribozyme. Ribozyme folding is complex with formation of both secondary and tertiary contacts resulting in a tight globular three-dimensional structure. Secondary folding and to some extent, tertiary contacts can form in the presence of monovalent ions. However, complete formation of tertiary folding of the ribozyme requires divalent cations such as magnesium, manganese and calcium. In one embodiment of the present invention, magnesium chloride was added at x mM, where x denotes a whole number between 1 and 20 millimolar. In a preferred embodiment, 10 mM magnesium chloride was added.

Also described herein is the method for cleavage (breaking of covalent bond) by the ribozyme to liberate mRNA for purification. Substrate-dependent ribozymes require specific substrates such as glucosamine, imidazole etc. for binding at the active site of the ribozyme and positioning to elicit specific cleavage on the RNA molecule[20-22]. In a preferred embodiment, the substrate was added at 1 mM or 10 mM final concentrations and further incubated for x minutes, where x is a whole number between 20 minutes and 120 minutes. Addition of magnesium ions and the specific substrate causes both folding and subsequent cleavage of the ribozyme respectively.

Also described herein is a method for elution (collection of purified material) after cleavage by the ribozyme. In some embodiments, the mRNA is eluted or separated by simply collecting off the tube under magnet or by flowing elution buffer through a chromatography column. In some embodiments, the NaCl concentration was reduced to less than 100 millimolar monovalent concentration for efficient separation of mRNA from the ribozyme as there still could be weakly electrostatic interactions between mRNA and the ribozyme above 100 millimolar monovalent ion concentration.

In one embodiment, the ribozyme is an imidazole dependent HDV ribozyme and in another embodiment, the ribozyme is a glucosamine (Glm) or glucosamine-6-phosphate (Glm-6P) dependent ribozyme[20-23]. Both cleave at the 5′ end of the ribozyme and generate a 2′-3′ cyclic phosphate at the 3′ end of the mRNA of interest.

Also described herein is a method of synthesis of mRNA as described in prior art and is known to be easily performed by a person with reasonable experience in the skill [11, 24-33]. It needs to be noted that the generation of mRNA is not limited to production by IVT alone but may also include production of mRNA using other methods such as the ones described in prior art[17, 34-36] as long as the mRNA-ribozyme-adapter is user-defined and encoded by means such as a DNA plasmid or DNA template.

In one embodiment, the method of IVT comprises the following reaction components. 1×T7 RNA polymerase buffer (NEB), 0.5 millimolar each of ATP, GTP, CTP and UTP, 500 nanogram total DNA template and 250 units of T7 RNA polymerase (NEB) in a 50 microliter IVT reaction. The reaction may be incubated at 37 degrees Celsius for time x, where x may indicate any time between 30 minutes and 120 minutes, while in a preferred embodiment the incubation time is 120 minutes. The template for IVT may be synthesized using a polymerase chain reaction (PCR) or linearized from a DNA plasmid, both facile processes that can be accomplished by someone familiar with the art.

In one embodiment, the method of IVT is followed by silica purification of mRNA using commercially available purification kits, before subjecting to chromatography. In another embodiment, the method of IVT is followed by application of mRNA directly to the chromatography matrix. In one embodiment, DNAse treatment was performed on the silica column following binding of RNA but before washing and elution. In another embodiment, DNAse treatment was performed in solution after the IVT reaction.

Also described herein is a method of regeneration of the resin or chromatography column in the presence of pure RNAse-free water which may or may not be followed by additional treatments. In one embodiment, purified RNAse-free water elutes remains of cleaved cistRNA and uncleaved cistRNA. In another embodiment, water elution is followed by additional regeneration step(s) such as treatment with 1×TE (10 mM Tris pH 7 and 1 mM EDTA) at temperature x, where x may indicate 25°, 37°, 42° and 65° C. Additional cleaning may involve NaOH at x concentration, where x may be 5, 15 mM or 20 mM and not 10 mM.

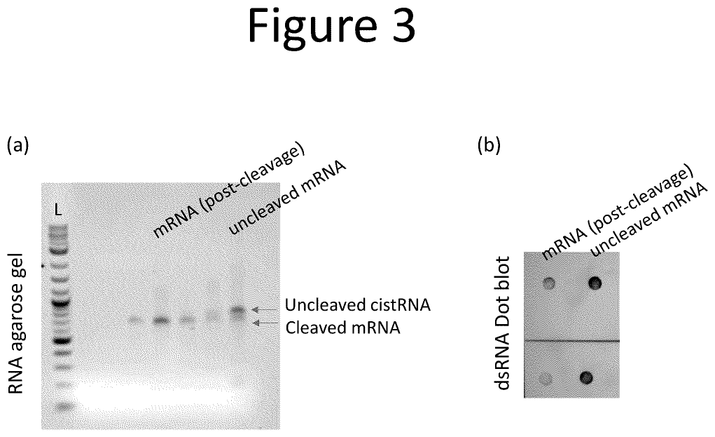

The results (FIGS. 3a and 3b) demonstrate that ‘SPECC’ can act as a general method of purification of mRNA. The said method of purification results in mRNA that is free of the uncleaved cistRNA (FIG. 3a) and shows substantial reduction in dsRNA (FIG. 3b).

This is the preferred embodiment of the invention. The technology to create this invention is listed as the preferred embodiment of this invention, but other methods are possible and are within the contemplation of this patent.

### REFERENCES

1. Gholamalipour, Y., A. Karunanayake Mudiyanselage, and C. T. Martin, 3′ end additions by T7 RNA polymerase are RNA self-templated, distributive and diverse in character-RNA-Seq analyses. Nucleic Acids Res, 2018. 46(18): p. 9253-9263.
2. Kapnick, S. M., The Nanoparticle-Enabled Success of COVID-19 mRNA Vaccines and the Promise of Microneedle Platforms for Pandemic Vaccine Response. DNA Cell Biol, 2022. 41(1): p. 25-29.
3. Mullard, A., COVID-19 vaccine success enables a bolder vision for mRNA cancer vaccines, says BioNTech CEO. Nat Rev Drug Discov, 2021. 20(7): p. 500-501.
4. Fuchs, A. L., A. Neu, and R. Sprangers, A general method for rapid and cost-efficient large-scale production of 5′ capped RNA. RNA, 2016. 22(9): p. 1454-66.
5. Garber, K., mRNA pioneers refocus on therapeutics. Nat Rev Drug Discov, 2022.
6. Dolgin, E., Startups set off new wave of mRNA therapeutics. Nat Biotechnol, 2021. 39(9): p. 1029-1031.
7. Rosa, S. S., et al., mRNA vaccines manufacturing: Challenges and bottlenecks. Vaccine, 2021. 39(16): p. 2190-2200.
8. Nelson, J., et al., Impact of mRNA chemistry and manufacturing process on innate immune activation. Sci Adv, 2020. 6(26): p. eaaz6893.
9. Webb, C., et al., Current Status and Future Perspectives on MRNA Drug Manufacturing. Mol Pharm, 2022. 19(4): p. 1047-1058.
10. Chen, J. and Q. Xu, Current Developments and Challenges of mRNA Vaccines. Annu Rev Biomed Eng, 2022.
11. Ouranidis, A., et al., mRNA Therapeutic Modalities Design, Formulation and Manufacturing under Pharma 4.0 Principles. Biomedicines, 2021. 10(1).
12. Baiersdörfer, M., et al., A Facile Method for the Removal of dsRNA Contaminant from In Vitro-Transcribed mRNA. Mol Ther Nucleic Acids, 2019. 15: p. 26-35.
13. Wilson, M. J., et al., Removal of tightly bound endotoxin from biological products. J Biotechnol, 2001. 88(1): p. 67-75.
14. Leamy, K. A., et al., Bridging the gap between in vitro and in vivo RNA folding. Q Rev Biophys, 2016. 49: p. e10.
15. Kumar, S. and T. Mohapatra, Deciphering Epitranscriptome: Modification of mRNA Bases Provides a New Perspective for Post-transcriptional Regulation of Gene Expression. Front Cell Dev Biol, 2021. 9: p. 628415.
16. Bodi, Z., et al., Yeast m6A Methylated mRNAs Are Enriched on Translating Ribosomes during Meiosis, and under Rapamycin Treatment. PLoS One, 2015. 10(7): p. e0132090.
17. Baronti, L., et al., A guide to large-scale RNA sample preparation. Anal Bioanal Chem, 2018. 410(14): p. 3239-3252.
18. ISSA, W., Joseph, J. AUNINS, Grant, and S. BANCEL, RIBONUCLEIC ACID PURIFICATION, E.P. OFFICE, Editor. 2018, ModernaTX, Inc.
19. Davis, J., J. Debear, and C. Cheng, Polynucleotide purification with monolith columns, W.I.P. Organization, Editor. 2017, Alexion Pharmaceuticals, Inc.
20. Kieft, J. S. and R. T. Batey, A general method for rapid and nondenaturing purification of RNAs. RNA, 2004. 10(6): p. 988-95.
21. McCarthy, T. J., et al., Ligand requirements for glmS ribozyme self-cleavage. Chem Biol, 2005. 12(11): p. 1221-6.
22. Soukup, G. A., Core requirements for glmS ribozyme self-cleavage reveal a putative pseudoknot structure. Nucleic Acids Res, 2006. 34(3): p. 968-75.
23. McCown, P. J., W. C. Winkler, and R. R. Breaker, Mechanism and distribution of glmS ribozymes. Methods Mol Biol, 2012. 848: p. 113-29.
24. Henderson, J. M., et al., Cap 1 Messenger RNA Synthesis with Co-transcriptional CleanCap. Curr Protoc, 2021. 1(2): p. e39.
25. Foster, J. B., D. M. Barrett, and K. Karikó, The Emerging Role of In Vitro-Transcribed mRNA in Adoptive T Cell Immunotherapy. Mol Ther, 2019. 27(4): p. 747-756.
26. Karikó, K., In vitro-Transcribed mRNA Therapeutics: Out of the Shadows and Into the Spotlight. Mol Ther, 2019. 27(4): p. 691-692.
27. Piao, X., et al., Double-stranded RNA reduction by chaotropic agents during. Mol Ther Nucleic Acids, 2022. 29: p. 618-624.
28. Loomis, K. H., et al., In Vitro Transcribed mRNA Vaccines with Programmable Stimulation of Innate Immunity. Bioconjug Chem, 2018. 29(9): p. 3072-3083.
29. Zhang, Y., et al., Chemically modified in-vitro-transcribed mRNA encoding thrombopoietin stimulates thrombopoiesis in mice. Mol Ther Nucleic Acids, 2022. 29: p. 657-671.
30. Zhang, Y., et al., Opportunities and Challenges for mRNA Delivery Nanoplatforms. J Phys Chem Lett, 2022. 13(5): p. 1314-1322.
31. Miliotou, A. N. and L. C. Papadopoulou, In Vitro-Transcribed (IVT)-mRNA CAR Therapy Development. Methods Mol Biol, 2020. 2086: p. 87-117.
32. Elkhalifa, D., et al., Chemically modified mRNA beyond COVID-19: Potential preventive and therapeutic applications for targeting chronic diseases. Biomed Pharmacother, 2022. 145: p. 112385.
33. Bhat, B., S. Karve, and D. G. Anderson, mRNA therapeutics: beyond vaccine applications. Trends Mol Med, 2021. 27(9): p. 923-924.
34. El Khouri, M., et al., Coexpression and Copurification of RNA-Protein Complexes in Escherichia coli. Methods Mol Biol, 2021. 2323: p. 67-73.
35. Ponchon, L. and F. Dardel, Purification of RNA expressed in vivo inserted in a tRNA scaffold. Methods Mol Biol, 2012. 941: p. 1-8.
36. Zemora, G. and C. Waldsich, RNA folding in living cells. RNA Biol, 2010. 7(6): p. 634-41.

1. A method of purifying mRNA comprising the following steps:
a. Application of a reaction mix or crude lysate containing mRNA-ribozyme-adapter to a column matrix in the presence of monovalent ions that allows binding by base-pairing of adapter to DNA oligo on the column;
b. Washing the column with a wash buffer to remove unbound contaminants;
c. Incubation with magnesium or another divalent ion(s) to allow folding of the ribozyme
d. Addition of substrate specific for the ribozyme and incubation for a specified duration to cleave off the mRNA;
e. Passive collection of mRNA following ribozyme cleavage in the presence of monovalent ions such that the base-pairing between the ribozyme and oligo-column is maintained but the mRNA is separated
f. Regeneration of the column by washing the column in the absence of monovalent and divalent ions such that the base-pairing and structure of RNA are disrupted and thus the ribozyme-adapter RNA segment is cleared off the column.

2. The method of claim 1 wherein the mRNA-ribozyme-adapter sequence contains the following: A sequence of mRNA comprising a UTR; an open reading frame (ORF), a 3′ UTR and a poly(A) tail, followed by a ribozyme and an adapter sequence.

3. The method of claim 1 wherein the synthesis of mRNA comprises the following steps
a. Synthesis of DNA template which encodes an RNA comprising an mRNA, a ribozyme and an adapter sequence
b. Synthesis of RNA using conventional cell-free, enzymatic IVT or overexpression in cells.
